(19) United States
(12) Patent Application Publication
(10) Pub. No.: US 2026/0325784 A1
(43) Pub. Date: Oct. 22, 2026

(54) Portable Pulse-Generating Device for Generating Low-Frequency Therapeutic Vibrations

(71) Applicant: Mason Schulze, Jr., Patterson Lakes (AU)

(72) Inventor: Mason Schulze, Jr., Patterson Lakes (AU)

(21) Appl. No.: 19/181,389

(22) Filed: Apr. 17, 2025

(51) Int. Cl. A61M 21/00

(52) U.S. Cl. CPC A61M 21/00; A61M 2021/0022; A61M 2021/0027; A61M 2021/0088

(57) ABSTRACT

A handheld wellness device includes a housing configured to be held in a user's hand, a vibration motor disposed within the housing and configured to generate vibration pulses within a frequency range of 5-40 Hz, a microcontroller unit electronically coupled to the vibration motor and configured to control pulse duration, vibration intensity, and session timing of the vibration motor, a user control interface for selecting operational modes, and a battery. The microcontroller unit is programmed with multiple operational modes, each corresponding to a different pattern of vibration pulses configured to induce stress relief, relaxation, sleep enhancement, and anxiety reduction. The device may include a sound driver unit producing audio output harmonized with the vibration pulses, an LED indicator for visual feedback, and a loop feature to secure the device to a user's hand or wrist. Methods of using the device include applying it to various body locations to transmit therapeutically effective vibration pulses.

### FIELD OF THE DISCLOSURE

The present invention relates to portable wellness devices, specifically to a handheld apparatus that generates controlled low-frequency vibration pulses. The invention particularly pertains to non-pharmaceutical methods for inducing parasympathetic nervous system responses, stress reduction, and sleep enhancement through tactile stimulation without requiring external components such as wearables, adhesives, or application-based controls.

### BACKGROUND OF THE INVENTION

In recent decades, stress and anxiety have emerged as significant global health concerns affecting millions of individuals across diverse demographics. The World Health Organization has recognized stress as a major contributor to numerous physical and mental health conditions, including cardiovascular disease, depression, and compromised immune function. As awareness of these issues has grown, so too has the demand for accessible, non-pharmaceutical interventions to manage stress and promote overall wellness.

Traditional approaches to stress management have included meditation, deep breathing exercises, and various forms of therapy. Meanwhile, technological solutions have evolved from simple mechanical devices to sophisticated digital applications. Early iterations of stress-relief technology included basic massage tools and vibrating devices that provided temporary physical relief but lacked specificity in targeting the body's natural relaxation mechanisms.

The therapeutic effects of vibration and tactile stimulation have been documented across numerous scientific studies. Research has demonstrated that specific frequencies of vibration, particularly those in the 5-40 Hz range, can influence physiological parameters including heart rate variability, cortisol levels, and respiratory patterns. These findings have catalyzed the development of various vibration-based wellness technologies.

Prior art in this field includes large, stationary vibration platforms primarily used in physical therapy settings, which, while effective, lack portability and accessibility for daily use. Wearable technologies subsequently emerged, offering continuous biometric monitoring and feedback through devices attached to the body with adhesives or straps. These wearables typically rely on companion mobile applications for operation, creating a technological barrier for many potential users, particularly older demographics or those in stress situations where complex technology interaction is impractical.

Another category of prior solutions includes vibrating cushions or mattresses designed for relaxation. While these provide some benefits, they are fundamentally immobile, limiting their utility to specific locations. Handheld massagers represent another approach, but these devices typically focus on muscle relaxation rather than nervous system regulation, often employing higher-frequency vibrations that may actually stimulate rather than calm the user.

More recent developments include sophisticated biofeedback devices that provide sensory guidance for breathing exercises. While innovative, these solutions typically require significant user training, ongoing engagement with digital interfaces, and often come at premium price points that limit accessibility.

A significant gap exists between simple mechanical devices lacking therapeutic precision and complex digital solutions requiring technical proficiency. This technological gap is particularly problematic given that stress responses often impair cognitive function, making complicated devices counterproductive during acute stress episodes. Furthermore, many existing solutions require multiple components, regular maintenance, or consistent access to charging and update capabilities.

Sleep technology represents another relevant domain, with numerous devices designed to improve sleep quality. However, most current solutions either monitor sleep without active intervention or require significant lifestyle adjustments and consistent usage patterns. The transition period between wakefulness and sleep—a critical window for many individuals with sleep difficulties—remains inadequately addressed by current technologies.

Perhaps most importantly, existing technologies frequently fail to provide immediate relief in situations of acute stress. Many require preparation, setup time, or specific environmental conditions that limit their effectiveness during unexpected stress episodes, such as panic attacks or anxiety surges.

The object of the present invention is therefore to provide a portable, user-friendly wellness device that delivers precise, therapeutically effective vibration patterns without requiring technical expertise, external components, or complex setup procedures. The invention aims to bridge the gap between simplistic mechanical devices and complex digital solutions, providing accessible stress-relief technology that can be immediately deployed in various contexts. Additionally, the invention seeks to address the transition to sleep through targeted parasympathetic nervous system activation, offering a non-pharmaceutical approach to sleep enhancement that operates independently of external systems or ongoing user engagement.

### SUMMARY OF THE INVENTION

In some aspects, the present invention provides a handheld wellness device designed to deliver controlled vibration pulses that induce physiological responses associated with relaxation and stress reduction. The device integrates multiple components within a compact housing that is specifically configured to be held comfortably in a user's hand, enabling convenient application during various situations requiring stress management or relaxation support.

Some aspects of the invention include a vibration motor disposed within the housing, carefully engineered to generate vibration pulses within a specific frequency range of 5-40 Hz. This frequency range has been identified through research as particularly effective in promoting parasympathetic nervous system activation, which is associated with the body's “rest and digest” state as opposed to the sympathetic “fight or flight” response.

In further aspects, the invention incorporates a sophisticated microcontroller unit that is electronically coupled to the vibration motor. This microcontroller serves as the central processing unit of the device, providing precise control over multiple vibration parameters including pulse duration, vibration intensity, and session timing to deliver optimized stimulation patterns.

Other aspects of the invention relate to the user control interface, which is strategically positioned on the exterior of the housing and electronically coupled to the microcontroller unit. This interface is designed with simplicity in mind, allowing users to easily select between operational modes even during periods of stress or anxiety when cognitive function may be temporarily impaired.

Further embodiments describe a self-contained power delivery system comprising a battery disposed within the housing and electrically coupled to both the microcontroller unit and the vibration motor. This configuration ensures that the device maintains complete portability without requiring external power sources or connections during operation.

Some aspects of the invention relate to the programmable nature of the microcontroller unit, which contains multiple pre-programmed operational modes. Each mode corresponds to a different pattern of vibration pulses, with patterns specifically designed to induce distinct physiological responses based on the user's current needs, whether for acute stress relief, meditation support, or sleep preparation.

In certain embodiments, the housing includes a soft silicone pad disposed on its outer surface, providing a comfortable tactile experience when the device is held or placed against the skin. This material selection enhances the therapeutic experience while also improving grip security during extended sessions.

Other aspects describe variations in the user control interface, including configurations with single or multiple button arrangements that allow for intuitive operation without requiring visual attention. This feature is particularly valuable during nighttime use or in situations where the user's attention is otherwise directed.

Further embodiments incorporate touch panel technology for the user control interface, offering an alternative interaction method that may provide additional functionality or accessibility benefits for certain users, while maintaining the device's fundamental simplicity.

In some implementations, the invention includes a sound driver unit disposed within the housing and electronically coupled to the microcontroller unit. This additional component expands the device's therapeutic capabilities by providing auditory stimulation to complement the tactile vibration experience.

Even further aspects describe the sophisticated integration of the sound driver unit with the vibration motor, wherein the sound driver is specifically configured to produce audio output at frequencies that are harmonized with the vibration pulses. This synchronization creates a multi-sensory experience that may enhance the overall effectiveness of the device in promoting relaxation.

Some embodiments include an LED indicator electronically coupled to the microcontroller unit, configured to provide visual feedback regarding device status, selected modes, and battery condition. This feature enables users to quickly verify operational parameters without requiring complex displays or interfaces.

Further aspects of the invention relate to power management features programmed into the microcontroller unit, including an automatic shutoff feature that deactivates the device after a predetermined period of operation. This feature conserves battery life while also providing a natural session limitation that aligns with typical relaxation or meditation practices.

In certain implementations, the device incorporates a loop feature attached to the housing, configured to secure the device to a user's hand or wrist. This design element enables passive use during sleep or extended relaxation sessions, where active holding might be impractical or disruptive.

Other aspects describe the specific configuration of the vibration motor to generate pulses that mimic the frequencies of human touch and regulated breathing rhythms. This biomimetic approach leverages the body's natural response to familiar and comforting stimuli.

The methodological aspects of the invention involve a process beginning with receiving user input through the control interface to select an operational mode, followed by microcontroller activation of the selected mode, generation of the appropriate vibration pattern, and application of the device to the user's body.

In some implementations, the method involves holding the device in the user's hand specifically during meditation sessions, creating a tactile focal point that can enhance mindfulness practices and attention regulation.

Further methodological aspects describe placing the device against specific body locations including the chest, collarbone, or pulse points, where vibration transmission may have enhanced physiological effects due to proximity to major blood vessels or nerve clusters.

Even further aspects of the inventive method involve securing the device to the user's body via the loop feature, enabling continuous operation during sleep without requiring active engagement or risking device displacement during natural sleep movements.

In the final aspects, the methodology encompasses the synchronization of audio output with vibration patterns when the device includes the sound driver component, creating a harmonized multi-sensory experience that may provide enhanced effectiveness for certain users or in specific application scenarios.

### BRIEF DESCRIPTION OF THE DRAWINGS

The accompanying drawings, which are incorporated in and constitute a part of this specification, illustrate exemplary embodiments of the handheld wellness device and, together with the description, serve to explain the principles of the invention. The drawings are provided for illustrative purposes only and are not drawn to scale. The figures depict various aspects of the device including the external housing configuration, internal component arrangement, user interface elements, and operational positioning on different body locations. Cross-sectional views highlight the spatial relationships between the vibration motor, microcontroller unit, sound driver, and power source within the compact housing. Additional illustrations demonstrate the device's ergonomic contours and the loop feature that enables hands-free operation. Together, these illustrations provide a comprehensive visual representation of the invention's structure and functionality. In the drawings:

FIG. 1 shows a person utilizing the handheld wellness device with a loop attachment during sleep, with vibration signals emanating from the device.

FIG. 2 depicts a person in a meditation posture using the handheld wellness device, which is emitting vibration signals calibrated for meditative states.

FIG. 3 presents a component layout view revealing the internal structure of the device, including the outer housing, controller, vibration motor, sound driver, control interface, battery, and LED indicator.

FIG. 4 provides a perspective view of the assembled handheld wellness device, highlighting the LED indicator, button control interface, and wrist/hand loop attachment.

### DETAILED DESCRIPTION OF PREFERRED EMBODIMENTS

The following description of preferred embodiments refers to the accompanying drawings, which illustrate specific embodiments of the invention. Other embodiments having different structures and operations do not depart from the scope of the present invention.

As used herein, the terms “comprising,” “including,” “containing,” “characterized by,” and grammatical equivalents thereof are inclusive or open-ended and do not exclude additional, unrecited elements or method steps. Other than in the operating examples, or where otherwise indicated, all numbers expressing quantities or parameters used in the specification and claims are to be understood as being modified by the term “about.” The terms “a” and “an” refer to one or more of the elements described, whereas “plurality” refers to two or more elements.

The handheld wellness device described herein provides novel solutions for inducing physiological responses through controlled vibration pulses within the 5-40 Hz frequency range. The invention incorporates a microcontroller unit that precisely controls vibration parameters, a user-friendly interface, and optional features including sound generation harmonized with vibrations. The following detailed description provides a comprehensive understanding of the various embodiments and aspects of the invention.

This invention addresses the challenges of providing accessible stress management and relaxation support without requiring technical expertise or external components. By leveraging specific vibration frequencies known to promote parasympathetic nervous system activation, the device offers immediate relief during stress episodes while supporting meditation and sleep enhancement applications.

The invention's ability to deliver precisely controlled vibration patterns in a portable, self-contained format represents a significant advancement in wellness technology, bridging the gap between simple mechanical devices and complex digital solutions while supporting diverse applications across various user demographics and environments.

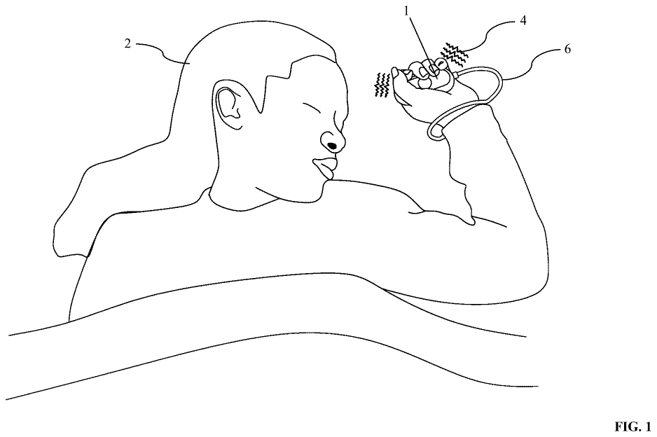

Firstly, the FIG. 1 illustrates an exemplary embodiment of the handheld wellness device in a sleep enhancement application. Firstly, the figure depicts a person (2) in a resting position while utilizing the handheld wellness device (1) secured to their wrist or hand. The compact device may be positioned in a manner that allows continuous operation throughout the sleep period without causing discomfort or requiring active engagement from the user.

In a non-limiting embodiment, the device (1) may be emitting vibration signals (4) that are specifically calibrated within the lower portion of the 5-40 Hz frequency range, which may be particularly conducive to promoting sleep onset and maintenance. These vibration patterns may mimic the natural rhythm of relaxed breathing, potentially encouraging the user's respiratory patterns to synchronize with the device output. The vibration signals (4) are visually represented in the figure as subtle wave patterns emanating from the device, though in actual operation these would be tactile rather than visible.

The embodiment of FIG. 1 further illustrates the loop feature (6) that may be incorporated into the housing design, enabling secure attachment to the user's wrist or hand during sleep. This loop (6) may be composed of an elastic material with sufficient flexibility to accommodate various user sizes while maintaining proper device positioning throughout natural sleep movements. The loop design may allow for blood circulation without restriction while preventing the device from becoming dislodged during typical sleep positional changes.

As shown in this implementation, the wellness device may continue to function autonomously throughout the sleep period, potentially utilizing one of the pre-programmed operational modes specifically designed for sleep enhancement. The microcontroller unit within the device may gradually adjust vibration parameters throughout the sleep session, potentially reducing intensity as sleep deepens or including an automatic shutoff feature after a predetermined duration. This configuration may be particularly beneficial for individuals experiencing difficulty with sleep onset or maintenance, providing a non-pharmaceutical intervention that requires minimal user interaction once initiated.

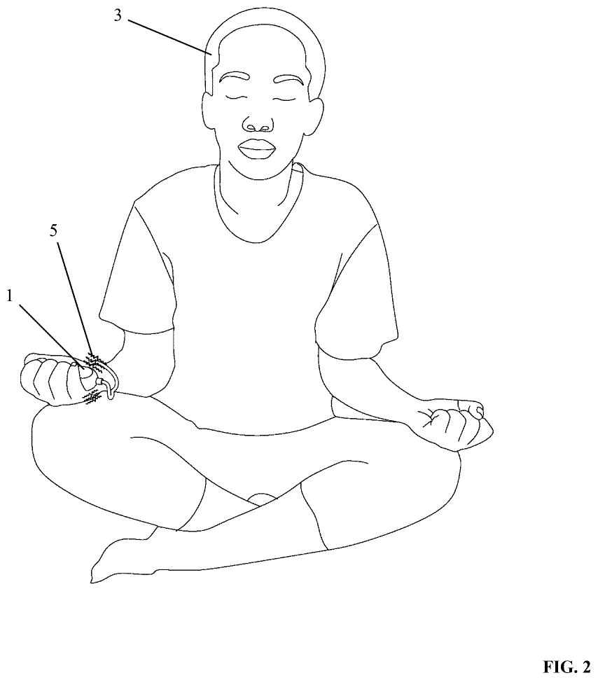

On the other hand, the FIG. 2 illustrates an exemplary embodiment of the handheld wellness device in a meditation application context. Firstly, the figure depicts a person (3) in a meditative posture while holding or placing the handheld wellness device (1) in a manner that facilitates mindfulness practice.

In a non-limiting embodiment, the device (1) may be generating vibration signals (5) that are specifically calibrated within the mid-range of the 5-40 Hz frequency spectrum, which may be particularly effective for promoting meditative states and sustained attention. These vibration patterns may be programmed to correspond with typical meditation intervals, potentially featuring rhythmic pulses that guide the user's breathing cadence or attention focus. The person (3) may be holding the device in their palm, placing it on their knee, or positioning it near their solar plexus—all positions that may effectively transmit the vibration signals (5) through the body. The compact nature of the device may allow for unobtrusive integration into various meditation practices, potentially serving as both a tactile anchor for attention and a physiological facilitator of parasympathetic nervous system activation through its precisely controlled vibration output.

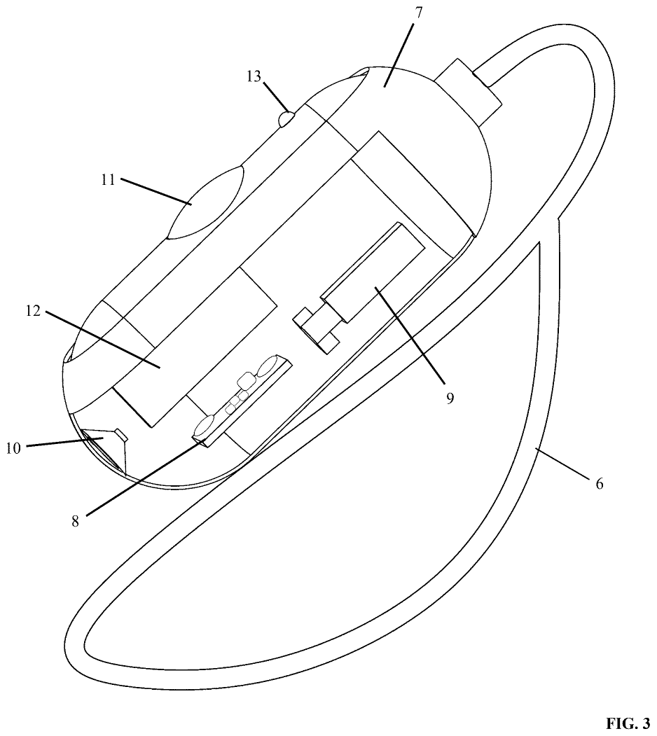

On the other hand, FIG. 3 provides a component layout view of an exemplary embodiment of the handheld wellness device, illustrating the spatial arrangement of the various functional elements within the compact structure. Firstly, the outer housing (7) may be observed encompassing all internal components, potentially constructed from durable ABS plastic with strategic silicone overlay portions that enhance grip comfort and tactile sensation when the device contacts the user's skin.

In a non-limiting embodiment, the controller (8) may be positioned centrally within the housing, serving as the computational core of the device. This microcontroller unit may be implemented as a compact circuit board containing necessary processing capabilities, memory storage for operational mode parameters, and interface connections to the other electronic components. The controller (8) may be programmed with multiple vibration patterns, each specifically designed to induce different physiological responses depending on the selected operational mode.

Secondly, the vibration motor (9) may be strategically positioned to optimize the transmission of vibration pulses through the housing to the user. This component may incorporate counterweights or eccentric mass configurations that generate the precisely controlled low-frequency vibrations within the 5-40 Hz range. The vibration motor (9) may be secured within the housing using vibration-dampening mounts that prevent unwanted resonance while directing the vibrational energy toward the contact surfaces.

The embodiment of FIG. 3 further includes a sound driver (10) that may be positioned adjacent to or integrated with the vibration motor (9). This audio component may incorporate a specialized transducer capable of producing low-frequency tones or white noise patterns that complement the vibration pulses. The sound driver (10) may be configured to synchronize its output with the vibration patterns, potentially creating harmonic relationships between tactile and auditory stimuli that enhance the overall parasympathetic response.

Additionally, the control interface (11) may be observed on an accessible portion of the outer housing, potentially implemented as one or more physical buttons, a touch-sensitive panel, or a combination thereof. This interface may be connected to the controller (8) via flexible circuitry that accommodates the compact design while ensuring reliable input detection. The control interface (11) may incorporate tactile feedback elements that confirm user inputs even when the device is not within visual range, such as during sleep applications.

Furthermore, the battery (12) may occupy a significant portion of the internal volume, potentially implemented as a rechargeable lithium-ion cell or other suitable power source. This component may be positioned to balance the device's weight distribution for optimal ergonomics while remaining accessible for replacement or recharging via a sealed port in the housing. The battery (12) may be selected to provide sufficient capacity for extended operation, potentially supporting 6-8 hours of continuous use between charging cycles.

Lastly, the LED indicator (13) may be positioned on a visible portion of the housing, potentially implementing one or more light-emitting elements that provide status information to the user. This component may display different colors or blinking patterns to communicate battery status, operational mode selection, or device status, serving as a simple visual interface that complements the tactile control system.

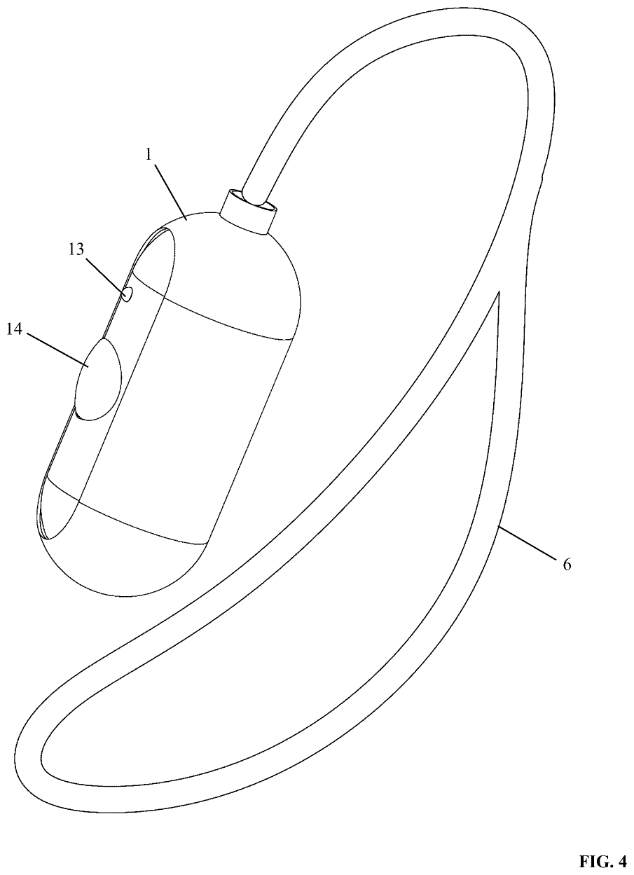

Further still, FIG. 4 illustrates a perspective view of an exemplary embodiment of the handheld wellness device (1), showcasing its external design features and user interface elements. Firstly, the device (1) may be observed in its complete assembled form, potentially exhibiting ergonomic contours that facilitate comfortable handling during various application scenarios.

In a non-limiting embodiment, the LED indicator (13) may be positioned on the upper surface of the device, potentially implementing a small light-emitting element that provides visual feedback regarding operational status. This indicator may utilize different colors or blinking patterns to communicate battery level, selected mode, or active status to the user. The button (14), representing one possible implementation of the control interface, may be strategically positioned for easy access by the thumb or forefinger when the device is held in the hand. This button may feature a slightly raised profile with tactile feedback elements that confirm successful activation even without visual confirmation. Additionally, the loop feature (6) may be observed extending from one end of the housing, potentially constructed from a flexible, durable material that allows the device to be secured to the user's wrist or hand during passive use scenarios such as sleep enhancement or extended relaxation sessions.

In some embodiments, the handheld wellness device may incorporate advanced haptic technology that allows for more complex vibration patterns beyond simple rhythmic pulses. This implementation may include multiple vibration motors positioned at different locations within the housing, enabling directional vibration effects that guide the user's attention or create wave-like sensations across the contact surface.

Another embodiment may feature biofeedback capabilities, where integrated sensors detect physiological parameters such as heart rate or skin conductance through contact points on the housing surface. The microcontroller may then adjust vibration patterns in real-time based on the detected parameters, potentially optimizing the device's effectiveness for each user's unique physiological response profile.

In certain implementations, the device may include expandable memory capabilities allowing users to download additional vibration programs through a connection to an optional companion application. This feature may enable personalization while maintaining the device's fundamental ability to function independently without requiring the application for standard operation.

Some alternative aspects may incorporate advanced power management systems including motion-activated operation, where the device remains in a low-power standby mode until movement is detected. This implementation may extend battery life significantly while ensuring the device is ready for immediate use during nighttime awakening or unexpected stress episodes.

Further embodiments may utilize alternative housing materials such as sustainably sourced wood or bamboo sections with antimicrobial properties, potentially offering both environmental benefits and enhanced hygiene for applications in healthcare settings. These natural materials may also provide different tactile experiences that some users might find more grounding or comforting than synthetic alternatives.

In another implementation, the device may incorporate temperature modulation capabilities, allowing for mild warming or cooling effects that complement the vibration therapy. This multisensory approach may enhance the overall relaxation response, particularly for users with specific temperature preferences or in environments with temperature extremes.

Some aspects may include a programmable timing feature that allows users to schedule automatic activation periods, potentially serving as a silent alarm alternative that wakes the user through gradually increasing vibration intensity rather than disruptive auditory alarms. This implementation may be particularly beneficial for individuals with hearing impairments or those who share sleeping spaces.

Alternative embodiments may feature modular attachment options beyond the standard loop, potentially including adhesive mounts, magnetic attachments, or wearable accessories that expand the device's application versatility. These attachments may enable secure positioning on various body locations or environmental surfaces without requiring active holding.

In certain implementations, the device may incorporate phototherapy elements, integrating soft LED lighting programmed to emit specific wavelengths associated with circadian rhythm regulation. This feature may be particularly beneficial for sleep applications or for use in environments with limited natural light exposure.

Another embodiment may utilize advanced materials science to create a housing with variable surface textures that can be adjusted by the user. This implementation might feature sections that can transition between smooth and textured surfaces through simple mechanical adjustments, accommodating different sensory preferences.

Some aspects may incorporate enhanced accessibility features such as raised tactile indicators, high-contrast visual elements, or vibration pattern variations specifically designed to communicate device status to users with visual impairments. These features may expand the device's usability across diverse user populations.

Finally, alternative implementations may include specialized versions optimized for specific applications such as pediatric anxiety management, featuring child-friendly housing designs and vibration patterns specifically calibrated for younger nervous systems. These specialized variants may maintain the core technology while adapting the form factor and interface to serve specific demographic needs.

### INDUSTRIAL APPLICATION

The handheld wellness device has applications in stress management, sleep enhancement, meditation support, and general wellness sectors. It provides a portable solution for individuals experiencing anxiety, insomnia, or stress-related conditions without requiring clinical settings. The device's industrial application extends to corporate wellness programs, healthcare facilities, meditation centers, and personal wellness markets. Its compact design and independent operation make it suitable for mass production with standardized components, allowing for cost-effective manufacturing and distribution. The device addresses the growing demand for non-pharmaceutical wellness solutions in an increasingly stress-prone global population, creating significant commercial potential across multiple industries.

What is claimed is:

1. A handheld wellness device comprising:
a housing configured to be held in a user's hand;
a vibration motor disposed within the housing and configured to generate vibration pulses within a frequency range of 5-40 Hz;
a microcontroller unit electronically coupled to the vibration motor and configured to control pulse duration, vibration intensity, and session timing of the vibration motor;
a user control interface disposed on the housing and electronically coupled to the microcontroller unit, the user control interface configured to receive user input for selecting operational modes of the device;
a battery disposed within the housing and electrically coupled to the microcontroller unit and the vibration motor to provide power thereto; and
wherein the microcontroller unit is programmed with a plurality of operational modes, each operational mode corresponding to a different pattern of the vibration pulses configured to produce therapeutically effective vibration patterns that induce a calming physiological response, stress relief, or sleep enhancement in the user by mimicking natural touch and breathing rhythms.

2. The handheld wellness device of claim 1, wherein the housing includes a soft silicone pad disposed on an outer surface thereof.

3. The handheld wellness device of claim 1, wherein the user control interface comprises a single or multiple button configuration.

4. The handheld wellness device of claim 1, wherein the user control interface comprises a touch panel.

5. The handheld wellness device of claim 1, wherein the device further comprises a sound driver unit disposed within the housing and electronically coupled to the microcontroller unit.

6. The handheld wellness device of claim 5, wherein the sound driver unit is configured to produce audio output at frequencies harmonized with the vibration pulses.

7. The handheld wellness device of claim 1, wherein the device further comprises an LED indicator electronically coupled to the microcontroller unit and configured to provide visual feedback of device status.

8. The handheld wellness device of claim 1, wherein the microcontroller unit is programmed with an automatic shutoff feature that deactivates the device after a predetermined period of operation.

9. The handheld wellness device of claim 1, wherein the device further comprises a loop feature attached to the housing configured to secure the device to a user's hand or wrist.

10. The handheld wellness device of claim 1, wherein the vibration motor is specifically configured to generate vibration pulses that mimic frequencies of human touch and regulated breathing rhythms.

11. A method of using a handheld wellness device for inducing a physiological response, the method comprising:
receiving, via a user control interface disposed on a housing of the handheld wellness device, a user input to select an operational mode;
activating, in response to the user input, a microcontroller unit to initiate the selected operational mode;
generating, via a vibration motor controlled by the microcontroller unit, therapeutically effective vibration pulses within a frequency range of 5-40 Hz according to a pattern corresponding to the selected operational mode, wherein the pattern is configured to mimic natural touch and breathing rhythms; and
applying the handheld wellness device to a user's body to transmit the vibration pulses to the user to induce a calming parasympathetic nervous system response for promoting stress reduction, anxiety relief, or sleep enhancement.

12. The method of claim 11, wherein applying the handheld wellness device comprises holding the device in the user's hand during a meditation session.

13. The method of claim 11, further comprising securing the device to the user's hand or wrist via a loop feature attached to the housing.

14. The method of claim 11, further comprising producing audio output via a sound driver unit disposed within the housing at frequencies harmonized with the vibration pulses.

15. The method of claim 11, further comprising automatically deactivating the handheld wellness device after a predetermined period of operation.

16. The method of claim 11, wherein the vibration pulses are generated according to a pattern configured to mimic frequencies of human touch and regulated breathing rhythms.
